SEQUENCE LISTING (4 sequences)

SEQ ID NO: 1 - DNA, 20 bp, Artificial Sequence
  Primer for library construction and amplification
aatgatacggcgaccaccga

SEQ ID NO: 2 - DNA, 21 bp, Artificial Sequence
  Primer for library construction and amplification
caagcagaagacggcatacga

SEQ ID NO: 3 - DNA, 20 bp, Artificial Sequence
  Primer for library capture and amplification
aatgatacggcgaccaccga

SEQ ID NO: 4 - DNA, 21 bp, Artificial Sequence
  Primer for library capture and amplification
caagcagaagacggcatacga

(19) United States
(12) Patent Application Publication
(10) Pub. No.: US 2024/0026457 A1
(43) Pub. Date: Jan. 25, 2024

(54) APPLICATION OF VARIATIONS IN NOTCH FAMILY GENES IN PREDICTING SENSITIVITY TO IMMUNE CHECKPOINT INHIBITOR THERAPY IN PATIENTS WITH SOLID TUMORS

(71) Applicant: SHANGHAI ORIGIMED CO., LTD., Shanghai (CN)

(72) Inventors: Kai WANG, Shanghai (CN); Lin ZHANG, Shanghai (CN); Feng LI, Shanghai (CN); Caiping CHEN, Shanghai (CN)

(21) Appl. No.: 17/800,464

(22) PCT Filed: Feb. 18, 2021
(86) PCT No.: PCT/CN2021/076761

§ 371 Date: Aug. 17, 2022

(30) Foreign Application Priority Data
Feb. 18, 2020 (CN) 202010098983.2

(51) Int. Cl. C12Q 1/6886; G16B 20/20

(52) U.S. Cl. CPC C12Q 1/6886; G16B 20/20; C12Q 2600/106; C12Q 2600/156; C12Q 2600/166

(57) ABSTRACT

The invention relates to the field of clinical molecular diagnostics, in particular to the application of NOTCH family gene variation in predicting the sensitivity of solid tumor patients to immune checkpoint inhibitor therapy and predicting the degree of tumor mutation load of solid tumor patients. This method is helpful to simplify the detection content, reduce the detection cost of patients, speed up the issuance time of detection report, and the detection of gene mutation status is more reliable.

### TECHNICAL FIELD

The present disclosure relates to the field of clinical and molecular diagnostics, in particular to the application of variation in NOTCH family genes in predicting sensitivity to immune checkpoint inhibitor therapy in solid tumor patients.

### BACKGROUND

Tumor immunotherapy has been developed in full swing. Immune checkpoint inhibitor (ICI) is a “star” drug in the field of cancer treatment in recent years, and has entered the first-line treatment of cancer. Although immune checkpoint inhibitors are effective, the overall objective response rate (ORR) is only about 20%. Thus how to accurately screen the beneficiaries has become an urgent problem for clinicians.

Three immunotherapy biomarkers, programmed death ligand 1 (PD-L1), tumor mutational burden (TMB) and microsatellite instability (MSI), which are approved by the FDA (The (American) Food and Drug Administration) or recommended by NCCN (National Comprehensive Cancer Network) guidelines, have their respective advantages and disadvantages. PD-L1 is the most widely used immunotherapy biomarker, and PD-L1 immunohistochemistry (PD-L1 IHC) assay has also been approved by the FDA as a companion diagnostic for pembrolizumab first-line medication. However, the results of multiple clinical trials showed that the predictive ability of PD-L1 expression on the efficacy of immunotherapy is not consistent. Some PD-L1 negative patients can still benefit from immunotherapy with sustained remission time not inferior to PD-L1 positive patients. TMB is also a recommended biomarker for immunotherapy. However, it is difficult to establish a consensus on the TMB threshold in view of the differences in the TMB algorithm of different companies or laboratories. MSI has been regarded as a key tumor biomarker, and the FDA has agreed to use drugs based on MSI status rather than histopathological type. However, microsatellite instability (MSI-H) occurs infrequently in tumors, and there are some limitations in clinical promotion. The most important point is that the existing study (including 11,348 cases of solid tumors) found that the overlap rate of positive PD-L1, high TMB and MSI-H is only 0.6%, suggesting that many potential immunotherapy beneficiaries would be elided if any one of biomarkers is used alone. Therefore, it is necessary to further explore the immunotherapy biomarker.

With the increasing application of next-generation sequencing in tumor precision therapy, it is found that gene mutations in specific somatic cells may affect immune function to a tumor or response to immunotherapy, which indicates that specific somatic gene mutations may be potential immunotherapy prediction factors. Epidermal growth factor receptor (EGFR) mutations and anaplastic lymphoma kinase (ALK) rearrangements are potential predictors of poor prognosis for ICI immunotherapy. It is found from a retrospective analysis that only 3.6% of these patients responded to ICI immunotherapy, while the response rate of EGFR wild-type and ALK-negative or unknown patients was 23.3%. These gene mutations as biomarkers still cannot cover all potential immunotherapy beneficiaries. There is still a need in the field for more efficient and accurate methods and tools to identify patients with solid tumor suitable for immune checkpoint inhibitor therapy. In addition, although a large number of randomized comparison studies and large-sample real-world studies have confirmed the correlation between TMB and immune efficacy, TMB still can only reflects the number of tumor mutations, but cannot indicate the statu of tumor microenvironment. In addition, TMB assay requires high technical platforms, long working period, and high cost, which restrict its clinical application.

### SUMMARY

In order to achieve the above objectives of the present disclosure, the following technical solutions are specially adopted.

In an aspect, the present disclosure provides a detection agent for predicting sensitivity to an immune checkpoint inhibitor therapy in a subject with a solid tumor, wherein the detection agent is a detection agent for detecting NOTCH gene variations;

the NOTCH gene is at least one selected from the group consisting of NOTCH1, NOTCH2, NOTCH3, and NOTCH4.

In an embodiment, the detection agent is a detection agent for detecting NOTCH gene variations at the nucleic acid level.

In an embodiment, the detection agent is a detection agent for detecting NOTCH gene variations at the protein level.

In an embodiment, the detection agent is a group of DNA probes whose sequence is complementary to the sequence of at least one gene in NOTCH1, NOTCH2, NOTCH3, and NOTCH4.

In an embodiment, the detection agent is a group of DNA probes whose sequence is complementary to the selected gene sequence from a gene sequence group consisting of Gene ID: 4851, Gene ID: 4853, Gene ID: 4854, and Gene ID: 4855.

In an embodiment, the immune checkpoint inhibitor is a PD-1 inhibitor and/or a PD-L1 inhibitor.

In an embodiment, the PD-1 inhibitor may further be one or more selected from the group consisting of Nivolumab (OPDIVO; BMS-936558), Pembrolizumab (MK-3475), Jembrolizumab, lambrolizumab, Pidilizumab (CT-011), Tereprizumab (JS001) and Ipilimumab.

In an embodiment, the PD-L1 inhibitor may further be one or more selected from the group consisting of Atezolizumab (MPDL3280A), JS003, Durvalumab, Avelumab, BMS-936559, MEDI4736, and MSB0010718C.

In an embodiment, the gene variation includes one or more variation types of point mutations, truncation mutations, amplification variations and fusion/rearrangement.

In an embodiment, the gene variation includes point mutations and truncation mutations.

In another aspect, the present disclosure provides a kit for predicting immune checkpoint inhibitor therapy sensitivity of the subject with solid tumors. The kit comprises the detection agent as described above.

In an embodiment, the kit further includes a DNA negative quality control and a DNA positive quality control, wherein the DNA negative quality control is selected from wild-type NOTCH1, NOTCH2, NOTCH3, NOTCH4 genes, and combinations thereof, and the DNA positive quality control is selected from known variants of NOTCH1, NOTCH2, NOTCH3, NOTCH4 and combinations thereof.

In an embodiment, the kit further includes a sample processing reagent, including at least one of sample lysis reagent, sample purification reagent and sample nucleic acid extraction reagent.

In an embodiment, the sample is at least one selected from the group consisting of blood, serum, plasma, cerebrospinal fluid, tissue or tissue lysate, cell culture supernatant, semen, and saliva samples from the subject with the solid tumor.

In another aspect, the present disclosure provides a method for predicting the sensitivity to an immune checkpoint inhibitor therapy of a subject with solid tumors. The method includes: a) using the DNA extracted from a sample to be detected for library preparation to obtain a gDNA library; b) hybridizing the gDNA library with a group of DNA probes for detecting NOTCH gene variations to obtain a hybridization product, wherein the NOTCH gene is at least one selected from the group consisting of NOTCH1, NOTCH2, NOTCH3, and NOTCH4; c) performing amplification and purification after the hybridization capture to obtain a captured gDNA library; d) sequencing the captured gDNA library to obtain targeted gDNA sequencing data; e) performing bioinformatic analysis of the gDNA sequencing data to obtain the NOTCH gene variation information.

In an embodiment, the detection agent is a group of DNA probes for at least one sequence selected from the following gene sequences: Gene ID: 4851, Gene ID: 4853, Gene ID: 4854, and Gene ID: 4855.

In an embodiment, the immune checkpoint inhibitor is a PD-1 inhibitor and/or a PD-L1 inhibitor.

In an embodiment, the PD-1 inhibitor may further be one or more selected from the group consisting of Nivolumab (OPDIVO; BMS-936558), Pembrolizumab (MK-3475), Jembrolizumab, lambrolizumab, Pidilizumab (CT-011), Tereprizumab (JS001) and Ipilimumab.

In an embodiment, the PD-L1 inhibitor may further be one or more selected from the group consisting of Atezolizumab (MPDL3280A), JS003, Durvalumab, Avelumab, BMS-936559, MEDI4736, and MSB0010718C.

In an embodiment, in a), the total amount of DNA extracted from the sample to be detected is between 50 ng and 500 ng.

In an embodiment, in a), the extracted DNA is subjected to nucleic acid fragmentation to obtain nucleic acid fragments for constructing the gDNA library.

In an embodiment, the gDNA library is constructed by following steps: end repair & A-tailing, adapter ligation and post-ligation cleanup, so as to obtain a purified adaptor-ligated product for the gDNA library preparation. Further, the reaction system and reaction procedures for end-repair & A-tailing are shown in Table 4 and Table 5 below, respectively; the reaction system and reaction procedure for adaptor ligation are shown in Table 6 and Table 7 below, respectively.

In an embodiment, the purified adaptor-ligated product is subjected to library amplification and purification to obtain a gDNA library. Further, the reaction system of the library amplification is shown in Table 8 below. The amplification reaction procedures are as follows: (1) initial-denaturation at 98° C. for 3 minutes; (2) denaturation at 98° C. for 20 seconds; (3) annealing at 60° C. for 15 seconds; (4) extension at 72° C. for 30 seconds; (5) final extension at 72° C. for 5 minutes; wherein the items (2)-(4) repeat 5-13 cycles.

In an embodiment, in b), for the hybridization product amplification, prepare an amplification reaction mixture and transfer 30 μL amplification reaction mixture into a 0.2 mL PCR tube containing the hybridization product, mix thoroughly by vortexing and centrifuge briefly, and then proceed to amplification in a PCR Thermal Cycler. The hybridization product amplification reaction procedure is assembled as follows: (1) initial-denaturation at 98° C. for 45 seconds; (2) denaturation at 98° C. for 15 seconds; (3) annealing at 60° C. for 30 seconds; (4) extension at 72° C. for 1 minute; (5) final extension at 72° C. for 1 minute; wherein the items (2)-(4) repeat 11-13 cycles.

In the present disclosure, by taking into account variations in the NOTCH gene family, the TMB degree of a patient with a solid tumor can be accurately predicted, and consequently population sensitive to ICI, avoiding blind medication, and improving the economic performance of ICI treatment. In the present disclosure, screening of the variation in the NOTCH family gene, as a biomarker for predicting the ICI-sensitive population in patients with a solid tumor results in a more accurate prediction compared with the co-mutation of other gene combinations. The variation in the NOTCH family gene used in the present disclosure can be used as independent predictive risk factors in practical applications and improves the detection efficiency. This method is conducive to simplifying the analyte, reducing the cost of detection for a patient, and accelerating output of detection reports. Compared with the PD-L1 immunohistochemistry method, which requires manual interpretation on immunohistochemistry images, and TMB, which requires artificial determination of thresholds, this method, in which detection of the status of gene mutation is adopted, is more reliable.

### BRIEF DESCRIPTION OF THE DRAWINGS

To illustrate the technical solutions in the embodiments of the present disclosure or in the prior art more clearly, the following briefly introduces the accompanying drawings required for describing the embodiments. The accompanying drawings in the following description show merely some embodiments of the present disclosure, and persons of ordinary skill in the art may still derive other drawings from these accompanying drawings without creative efforts.

FIG. 1 shows the results of detection of frequency of NOTCH family genes (NOTCH′, NOTCH2, NOTCH3, and NOTCH4) in various types of tumors in an embodiment of the present disclosure;

FIG. 2 shows the mutation ratios of NOTCH1, NOTCH2, NOTCH3 and NOTCH4 in solid tumors in China in an embodiment of the present disclosure;

FIG. 3 shows the mutation frequency of different variant forms of NOTCH family genes (NOTCH1, NOTCH2, NOTCH3, and NOTCH4) in various types of tumors in an embodiment of the present disclosure;

FIG. 4 shows the effect of NOTCH family genes (NOTCH1, NOTCH2, NOTCH3, and NOTCH4) on TMB in all types of tumors in an embodiment of the present disclosure;

FIG. 5 shows the effect of NOTCH1, NOTCH2, NOTCH3, and NOTCH4 gene on TMB in all types of tumors in an embodiment of the present disclosure;

FIG. 6 shows the effect of point mutations or truncation mutations in NOTCH1, NOTCH2, NOTCH3, and NOTCH4 in patients on TMB in all tumor types in an embodiment of the present disclosure;

FIG. 7 is the analysis of mutation sites in NOTCH1, NOTCH2, NOTCH3, and NOTCH4 genes in an embodiment of the present disclosure;

FIG. 8 is comparison of the efficacy of receiving immunotherapy using immune checkpoint inhibitors in patients having NOTCH1, NOTCH2, NOTCH3, or NOTCH gene mutations with in wild-type patients in an embodiment of the present disclosure;

FIG. 9 shows Cox multi-factor analysis in an embodiment of the present disclosure, suggesting that NOTCH family gene mutation is an independent prognostic risk factor for immunotherapy;

FIG. 10 shows the relationship between the number of NOTCH family gene mutations and TMB in an embodiment of the present disclosure;

FIG. 11 shows the relationship between the number of NOTCH family variant genes and the effect of immunotherapy in an embodiment of the present disclosure;

FIG. 12 shows Cox multi-factor analysis in an embodiment of the present disclosure, suggesting that the number of NOTCH family variant genes is an independent prognostic risk factor for immunotherapy.

### DETAILED DESCRIPTION OF THE EMBODIMENTS

A reference to the embodiments of the present disclosure will now be provided in detail, one or more examples of which are described below. Each example is provided as an explanation of rather than a limitation to the disclosure. In fact, it is obvious to those skilled in the art that various modifications and changes can be made to the present disclosure without departing from the scope or spirit of the present disclosure. For example, features illustrated or described as part of one embodiment can be used in another embodiment to produce a still further embodiment.

Therefore, it is intended that the present disclosure covers such modifications and changes that fall within the scope of the appended claims and their equivalents. Other objects, features, and aspects of the present disclosure are disclosed in or are obvious from the following detailed description. Those of ordinary skill in the art should understand that the discussion is only a description of exemplary embodiments and is not intended to limit the broader aspects of the present disclosure.

The present disclosure relates to the application of a detection agent for NOTCH family gene variation in preparing a kit for predicting sensitivity to an immune checkpoint inhibitor therapy in a subject with a solid tumor, wherein the presence of the NOTCH family gene variation is an indication that the subject with the solid tumor is sensitive to immune checkpoint inhibitor therapy.

The present disclosure relates to the application of a detection agent for NOTCH family gene variation in preparing a kit for predicting a degree of tumor mutation burden in a subject with a solid tumor, wherein the presence of the NOTCH family gene variation is an indication of high tumor mutation burden.

In some embodiments, the solid tumor subject is a mammal.

In some embodiments, the solid tumor subject is a primate.

In some embodiments, the solid tumor subject is human, that is, a patient with a solid tumor.

In the present disclosure, the NOTCH family is at least one selected from NOTCH1, NOTCH2, NOTCH3, or NOTCH4, unless particularly emphasized. In some embodiments, the subject with the solid tumor is human; and the NOTCH family is as follows: NOTCH1: Gene ID: 4851, NM_017617.5; NOTCH2: Gene ID: 4853, NM_024408.4; NOTCH3: Gene ID: 4854, NM_000435.3; or NOTCH4: Gene ID: 4855, NM_004557.4.

As used herein, the term “immune checkpoint” refers to some inhibitory signaling pathways that exist in the immune system. Under normal circumstances in a body, immune checkpoints can maintain immune tolerance by regulating the intensity of autoimmune responses; however, when the body is invaded by tumors, the activation of immune checkpoints will cause inhibition of autoimmunity, which is conducive to the growth and escape of tumor cells. By using immune checkpoint inhibitors, the normal anti-tumor immune response in a body can be restored, thereby controlling and eliminating tumors.

In the present disclosure, the immune checkpoints include, but are not limited to, programmed death receptor 1 (PD-1), PD-L1, and cytotoxic T lymphocyte-associated antigen 4 (CTLA-4), as well as some newly discovered immune checkpoints such as lymphocyte activation gene 3 (LAG3), CD160, T-cell immunoglobulin and mucin-3 (TIM-3), V-domain Ig suppressor of T cell activation (VISTA), adenosine A2a receptor (A2aR), and the like.

Preferred immune checkpoint inhibitors are PD-1 inhibitors and/or PD-L1 inhibitors.

The PD-1 inhibitor may further be one or more selected from the group consisting of Nivolumab (OPDIVO; BMS-936558), Pembrolizumab (MK-3475), Jembrolizumab, lambrolizumab, Pidilizumab (CT-011), Tereprizumab (JS001) and Ipilimumab.

The PD-L1 inhibitor may further be one or more selected from the group consisting of Atezolizumab (MPDL3280A), JS003, Durvalumab, Avelumab, BMS-936559, MEDI4736, and MSB0010718C.

The terms “mutation burden” and “mutational burden” are used interchangeably herein. In the context of tumors, the mutational burden is also referred to herein as “tumor mutation burden”, “tumor mutational burden” or “TMB”.

In the present disclosure, the gene variation may include point mutations and fragment mutations. The point mutations may include single nucleotide polymorphisms (SNPs), base substitutions, single base insertions or base deletions, or silent mutations (for example, synonymous mutations). The fragment mutations can include insertion mutations, truncation mutations, or gene rearrangement mutations.

In some embodiment, the gene variation includes one or more of point mutations, truncation mutations, amplification variations, and fusion/rearrangement. More preferably, the gene variation includes point mutations and truncation mutations.

In some embodiments, mutations are located at nucleotides 263-7930 of NOTCH1 gene sequence (Gene ID: 4851), at nucleotides 257-7672 of NOTCH2 gene sequence (Gene ID: 4853), at nucleotides 917056 of NOTCH3 gene sequence (Gene ID: 4854), and at nucleotides 1406151 of NOTCH4 gene sequence (Gene ID: 4855).

In some embodiments, evaluation of gene variation of the NOTCH family gene includes determining whether there are point mutations/truncation mutations in the genes (e.g., coding regions).

In some embodiments, after a mutation in the coding region of the NOTCH family gene is determined, the expression of the NOTCH family gene, for example, the protein expression level of at least one of NOTCH1, NOTCH2, NOTCH3, and NOTCH4, is evaluated.

In some embodiments, the patient with the solid tumor is 40 to 80 years old, such as 50, 60, or 70 years old.

In some embodiments, the solid tumor includes tumors arising from any one or more lesions in bone, bone junction, muscle, lung, trachea, heart, spleen, artery, vein, capillary, lymph node, lymphatic vessel, lymphatic fluid, oral cavity, pharynx, esophagus, stomach, duodenum, small intestine, colon, rectum, anus, appendix, liver, gallbladder, pancreas, parotid gland, sublingual gland, urinary kidney, ureter, bladder, urethra, ovary, fallopian tube, uterus, vagina, vulva, scrotum, testis, vas deferens, penis, eyes, ears, nose, tongue, skin, brain, brain stem, medulla oblongata, medulla spinalis, cerebrospinal fluid, nerve, thyroid, parathyroid, adrenal gland, pituitary, pineal gland, pancreatic islets, thymus, gonad, sublingual gland and parotid gland.

In some embodiments, the solid tumor includes one or more of lung adenocarcinoma, lung squamous cell carcinoma, small cell lung cancer, hepatocellular carcinoma, esophageal tumor, breast cancer, head and neck cancer, endometrial cancer, and skin squamous cell carcinoma.

The NOTCH family gene is a gene that encodes a protein, with mutations of the gene usually also expressed at the transcription level and response level. Therefore, tests can be performed at the RNA and protein levels by those skilled in the art to indirectly reflect whether the NOTCH family gene has gene mutation. These can all be applied to the present disclosure.

In some embodiments, the detection agent is for detecting at the nucleic acid level.

The detection agent for detecting at the nucleic acid level (DNA or RNA level) can be selected from reagents well known to those skilled in the art, for example, a fluorescent-labeled nucleic acid (usually a probe or primer) that can hybridize to the DNA or RNA of a NOTCH gene family member. Moreover, it can be easily envisaged by those skilled in the art to reverse transcribe mRNA into cDNA and then detect the cDNA. Conventional replacement of these technical means is within the protection scope of the present disclosure.

In some embodiments, the detection agent is used to perform any of the following methods:
restriction fragment length polymorphism method, single-strand conformation polymorphism method, polymerase chain reaction, competitive allele-specific PCR, denaturing gradient gel electrophoresis, allele-specific PCR, nucleic acid sequencing, nucleic acid typing on-chip assay, flight mass spectrometry analysis, denaturing high performance liquid chromatography, Snapshot method, Taqman probe method, in situ hybridization, biological mass spectrometry and HRM method.

In some embodiments of the present disclosure, the nucleic acid sequencing may be transcriptome sequencing or genome sequencing. In some other embodiments of the present disclosure, the nucleic acid sequencing is high-throughput sequencing, also called next-generation sequencing (“NGS”). Next-generation sequencing generates thousands to millions of sequences simultaneously during a parallel sequencing. NGS is different from “Sanger sequencing” (first-generation sequencing) which is based on the chain termination products in a single sequencing reaction are separated by electrophoresis. The NGS sequencing platform that can be used in the present disclosure is commercially available and includes but is not limited to Roche/454 FLX, Illumina/Solexa GenomeAnalyzer, Applied Biosystems SOLID system, and the like. Almost all transcripts and gene sequences can also be quickly and comprehensively obtained for a specific cell or tissue of a species in a certain state through transcriptome sequencing on the second-generation sequencing platform, and transcriptome sequencing can be used to study gene expression, function, structure and alternative splicing of a gene, and prediction for a new transcript, etc.

In some embodiments, the detection agent is for detecting at a protein level.

In some embodiments, the detection agent is used to perform any of the following methods:
biological mass spectrometry, amino acid sequencing, electrophoresis, and detection with specific antibodies designed for mutation sites.

The detection with specific antibodies designed for the mutation site can further include immunoprecipitation, co-immunoprecipitation, immunohistochemistry, ELISA, Western Blot, etc.

In some embodiments, the kit further includes a sample processing reagent; furthermore, the sample processing reagent includes at least one of a sample lysis reagent, a sample purification reagent, and a sample nucleic acid extraction reagent.

In some embodiments, the sample is at least one selected from the group consisting of blood, serum, plasma, cerebrospinal fluid, tissue or tissue lysate, cell culture supernatant, semen, and saliva samples of the patient with the solid tumor.

In some embodiments, the tissue is cancerous tissue or para-cancerous tissue.

The sample for detection can also be selected from blood, serum, or plasma, which, in some embodiments, are derived from peripheral blood.

According to another aspect, the present disclosure provides a method for predicting sensitivity to an immune checkpoint inhibitor therapy in a patient with a solid tumor, comprising:

The detection agent as described above is used to determine whether or not a variation is in the NOTCH family gene.

The ideal scenario for diagnosis would be a situation wherein a single event or process would cause respective disease, e.g., in infectious diseases. In all other cases, it could be very difficult to make a correct diagnosis, especially when the disease cannot be fully and etiologically understood as is the case with many cancer types. As the skilled artisan will appreciate, no biochemical marker is diagnostic with 100% specificity and at the same time 100% sensitivity for a given multifactorial disease. Rather, biochemical markers, e.g., NOTCH family gene variation, can be used to assess with a certain likelihood or predictive value e.g., the presence, absence, or the severity of a disease. Therefore, in routine clinical diagnosis, generally various clinical symptoms and biological markers are took into account in the diagnosis for treatment and management of the underlying disease.

In some embodiments, the method is used for prognostic evaluation for the patient with a solid tumor after being subject to immune checkpoint inhibitor therapy.

The embodiments of the present disclosure will be described in detail below in conjunction with the Examples.

### EXAMPLES

The specific main components of the reagents used in the following examples of the present disclosure are shown in table 1.

TABLE 1

| name of the Reagent | Main components |
|---|---|
| End-Repairing Mix | Tris-HCl, MgCl₂, DTT, dNTP, ATP, Taq |
| | DNA polymerase, T4 DNA polymerase, |
| | and T4 polynucleotide kinase |
| Ligase buffer | Tris-HCl, MgCl₂, DTT, and ATP |
| Ligase | T4 DNA ligase |
| Primers for library | Oligonucleotide, and TE Buffer |
| construction | |
| and amplification | |
| Buffer for library | DNA polymerase, dNTPs, and MgCl₂ |
| construction | |
| and amplification | |
| Blocking agent 1 | Oligonucleotide |
| Blocking agent 2 | Human Cot-1 DNA, and Tris EDTA |
| Hybridization | Sodium phosphate buffer, SDS, EDTA, SSC, |
| buffer 1 | Denhardt's solution, and tetramethylammonium |
| | chloride |
| Hybridization | Polysucrose, polyvinylpyrrolidone, bovine |
| buffer 2 | serum albumin, and formamide |
| Probe library | Oligonucleotide |
| Washing buffer for | Tris-HCl, NaCl, EDTA, and Tween-20 |
| magnetic beads | |
| Washing buffer 1 | SSC, and SDS |
| Washing buffer 2 | SSC, and SDS |
| Washing buffer 3 | SSC |
| Washing buffer 4 | ethylenediamine tetraacetic acid disodium salt |
| Buffer for library | DNA polymerase, dNTPs, MgCl₂, and |
| capture and amplification | stabilizer |
| Primers for library | Oligonucleotides, and TE Buffer |
| capture and amplification | |
| DNA negative quality | Wild-type cell line DNA, and TE Buffer |
| control | |
| DNA positive quality | DNA of cell line with NOTCH1, NOTCH2, |
| control | NOTCH3, NOTCH4 variations, and TE Buffer |

The research methods adopted in the examples of the present disclosure are as follows:

Comprehensive Genome Analysis

Formalin-fixed, paraffin-embedded (FFPE) tissue samples of Chinese patients with solid tumors and control samples of paired peripheral whole blood were studied. Informed consent was signed by all patients. Targeted capture next-generation sequencing (NGS) was performed on OrigiMed, involving a combination of 450 cancer-related genes. DNA was extracted from unstained FFPE sections with a minimum of 20% tumor cellularity using QIAamp DNA FFPE Tissue Kit (QIAGEN) and from whole blood using QIAamp DNA Mini Kit (QIAGEN) and quantified with Qubit dsDNA HS assay kit (Thermo Fisher). After fragmented to ˜250 bp by sonication, DNA fragments were used for library construction with KAPA HyperPrep Kit followed by PCR amplification and quantification with Qubit dsDNA HS assay kit (Thermo Fisher).

A specific probe pool used for library targeted capture enrichment targeted ˜2.6 Mb of the human genome including 450 cancer-related genes and introns of genes rearranged frequently in cancer. All the candidate probes were designed according to the sequence information of the cancer-related gene exons and the rearrangement-related introns, then filtered by high specificity screening. The candidate probe fragments were aligned with the whole genome using BLAST. The probes are screened with the parameter E value of less than e 2° and with the number for which each of the probes pairing with other regions is less than 20, so as to ensure the comprehensiveness and specificity of the probes, thereby screening the probe library. E value indicates the likelihood that the similarity between other sequences and the target sequence is greater than the similarity between this sequence and the target sequence in the case of randomness. Thus, the lower the value is, the higher the reliability of the sequence alignment result is. In the present probe design, a stricter threshold of E value, i.e., less than e⁻²⁰, was used. In addition, the probe sequence is a shorter sequence which is only 120 bp long, so it may pair with other regions many times when aligning with whole genome. In order to ensure the specificity of the alignment, less number for which the probe sequence pairing with other regions should be better. Here, A stricter threshold for NGS probes, i.e., less than 20, is selected as the number of the probe sequence that pairs with other regions. Using the stricter threshold for the present probe design, high specificity of the probe may still be ensured, also showing that the design of the probe library involved in this example has a good effect.

The captured libraries were pooled, denatured and diluted to 1.5 to 1.8 pM, then performed paired-end sequencing on Illumina NextSeq 500 according to the manufacturer's protocol.

Three primer pairs that designed to amplify targeted regions in ACTIN gene were used for the sample DNA quality assessment:

i)
5′-CACACTGTGCCCATCTATGAGG-3′
and
5′-CACGCTCGGTGAGGATCTTC-3′,
ii)
5′-CACACTGTGCCCATCTATGAGG-3′
and
5′-TCGAAGTCCAGGGCAACATAGC-3′,
and
iii)
5′-CACACTGTGCCCATCTATGAGG-3′
and
5′-AAGGCTGGAAGAGCGCCTCGGG-3′.

The amplicon size of the three primer pairs described above is 100 bp, 200 bp and 300 bp, respectively. The sample DNA can be determined as passing quality assessment when all of the three targeted amplicons can be identified successfully.

Specifically, in the detection method of this example, the detection reagents involved are shown in table 2.

TABLE 2

| Number of | | | |
|---|---|---|---|
| Components | Name of Components | | Function |
| 1 | End Repair Mixture | | Construction of |
| 2 | Ligation buffer | | gDNA library |
| 3 | Ligase | | |
| 4 | Library Construction | | |
| | Amplification Primers | | |
| 5 | Library Construction | | |
| | Amplification mixture | | |
| 6 | Blocking agent 1 | | obtaining captured |
| 7 | Blocking agent 2 | | gDNA library |
| 8 | Hybridization buffer 1 | | |
| 9 | Hybridization buffer 2 | | |
| 10 | Probe pool | | |
| 11 | Other | Magnetic bead | |
| | reagents | washing buffer s | |
| 12 | | Washing buffer 1 | |
| 13 | | Washing buffer 2 | |
| 14 | | Washing buffer 3 | |
| 15 | | Washing buffer 4 | |
| 16 | | Captured Library | |
| | | Amplification Mixture | |
| 17 | | Captured Library | |
| | | Amplification Primers | |
| 18 | | Negative quality | Negative and positive |
| | | control DNA | quality controls for |
| 19 | | Positive quality | monitoring whether the |
| | | control DNA | entire process is normal |

In the detection method descried above, FFPE samples were used for detection. Samples with different types were processed as follows.

Unstained Sections:

1. Unstained sections had a thickness of 4-5 μm and a surface area greater than 1 cm². For a surgical tissue, 15 serial sections were prepared, and for a biopsy tissue, 25 serial sections were prepared.

Baking time was controlled within 10-15 minutes. Each of the sections should be marked with the pathology accession number.

2. FFPE blocks/sections with high tumor content and no necrotic tissue were selected for detection.

Fresh Surgical Tissue:

1. The surgical tissue had a size between 0.5×0.5×0.5 cm³ and 2×2×2 cm³.

2. During sampling, an area with abundant tumor tissue was selected, avoiding necrosis and ulcer tissues.

3. The tissues were placed in a surgical tissue sample tube in the test kit as soon as possible (within 10 minutes) after the tissues were isolated.

Biopsy Tissue:

1. For needle biopsy tumor samples, 2 or more pieces of the sample with a diameter ≥1 mm and a length ≥10 mm were collected.

2. For other biopsy tumor tissues, at least 2 pieces of samples which size was larger than millet were collected.

3. Re-biopsy was recommended if the biopsy tumor tissue contains too much necrosis tissues,

4. The tissues were placed in a biopsy tissue sample tube in the test kit as soon as possible (within 10 minutes) after the tissues were isolated.

The detection method specifically includes the following steps:

Step (I), obtain the library: DNA extracted from the sample to be tested was used for preparation and amplification to obtain the gDNA library. The library preparation steps are as follows:

1. Nucleic Acid Extraction

Nucleic acid was extracted from FFPE samples and blood controls according to the instructions with matched DNA extraction kits. Proceeded to quantify the extracted gDNA using Qubit™ dsDNA HS Assay Kit with matched instrument. The total amount of gDNA should be ≥50 ng. For gDNA extracted from FFPE samples, targeted fragments of the three ACTIN gene primer pairs must be identified after amplification. The gDNA should be stored at −25° C. to −15° C. if the next step was not proceeded immediately.

2. Nucleic Acid Fragmentation

The nucleic acid fragmentation aims to obtain about 300 bp nucleic acid molecular fragments. Fragmentation by sonication was suggested, and Covaris LE220-plus platform was recommended.

2.1 gDNA Fragmentation

5 μL of DNA negative quality control and 54, of DNA positive quality control in the kit were used for detection and processed with samples to be tested at the same time. 500 ng of gDNA was used if the total gDNA amount was equal to or greater than 500 ng; and all of the gDNA was used if the total gDNA amount was equal to or greater than 50 ng but less than 500 ng. If the volume of gDNA solution is less than 50 μL, bring the volume up to 50 nL with TE Buffer.

2.1.2 The gDNA sample to be fragmented was mixed by vortexing and centrifuge briefly then transferred into a microTUBE.

2.1.3 Fragmentation was performed according to the conditions shown in table 3.

TABLE 3

| Base | Peak | | Cycles | Treat- | | |
|---|---|---|---|---|---|---|
| Pair | Incident | Duty | per | ment | Temper- | Sample |
| Mode | Power | Factor | Burst | time | ature | Volume |
| (bp) | (W) | (%) | (cpb) | (sec) | (° C.) | (μL) |
| 300 | 450 | 30 | 200 | 80 | 7 | 50 |

2.1.4 After fragmentation, centrifuge the microTUBE, and transfer all the fragmented solution into a new labeled 0.2 mL PCR tube.

3. Library Construction

3.1 End Repair and A-Tailing

3.1.1 The End-Repair Mixture was thawed and mix by inversion. Assemble the reaction system according to table 4 in the 0.2 mL PCR tube.

TABLE 4

| Component | Volume |
|---|---|
| Fragmented nucleic acid (product of previous nucleic acid | 50 μL |
| fragmentation) | |
| End-Repair Mixture | 15 μL |
| Total Volume | 65 μL |

3.1.2 Mix by gentle pipetting up and down. Do not mix by vortexing. Centrifuge briefly to ensure the content is collected at the bottom of the tube. Reaction was performed according to the procedure shown in table 5.

TABLE 5

| Temperature | Time |
|---|---|
| Heated Lid (105° C.) | Turn on |
| 20° C. | 15 min |
| 65° C. | 15 min |
| 4° C. | Hold |

3.2 Adapter Ligation

3.2.1 The Ligation buffer was thawed, homogenized by inversion, and placed on ice on standby.

3.2.2 The reaction system was prepared according to table 6 in the 0.2 mL PCR tube in which end repair and A-tailing was performed.

TABLE 6

| Component | Volume | |
|---|---|---|
| End Repair and A-tailing Reaction Product | 65 | μL |
| Ligation buffer | 25 | μL |
| Ligase | 5 | μL |
| Adapter* | 2 | μL |
| Nuclease-Free Water | 3 | μL |
| Total Volume | 100 | μL |

*IDT UDI Adapter Kit was recommended for use.

3.2.3 The system was mixed by gentle pipetting up and down. Do not mix by vortexing. Centrifuge briefly to ensure the content is collected at the bottom of the tube. Reaction was performed according to the procedure shown in Table 7.

TABLE 7

| Temperature | Time |
|---|---|
| Heated Lid (105° C.) | Turn on |
| 20° C. | 15 min |
| 4° C. | Hold |

3.3 Post-Ligation Purification

Agencourt AMPure XP Beads were recommended for purification.

3.3.1 Magnetic beads were equilibrated at room temperature at least 30 minutes and thoroughly homogenized by vortexing, and transfer 80 μL of beads into a new 1.5 mL centrifuge tube.

3.3.2 100 μL of the ligation product was transferred into the 1.5 mL centrifuge tube prepared in step 3.3.1, mixed by vortexing, and incubated at room temperature for 5 min.

3.3.3 Place the 1.5 mL centrifuge tube on a magnetic rack until the solution is completely clear, remove and discard the supernatant (Do not disturb magnetic beads).

3.3.4 200 μL of freshly prepared 80% ethanol was added, incubate at room temperature for sec, and then remove and discard the supernatant.

3.3.5 Step 3.3.4 was repeated once.

3.3.6 Centrifuge briefly and place the 1.5 mL centrifuge tube on the magnetic rack and incubate for 1 min, and remove all residual solution, and open the lid for drying the beads at room temperature until ethanol has been completely evaporated. (Do not over dry the beads, which may result in reduced yield when beads are cracked.)

3.3.7 Resuspend dried beads with 22 μL of Nuclease-Free Water, mix thoroughly until homogenized, incubate at room temperature for 2 min, centrifuge briefly and place the tube on the magnetic rack. After the solution was completely clear, 20 μL of supernatant was transferred into a new labeled 0.2 mL PCR tube.

3.4 Library Amplification Processing

3.3.1 Mix the library construction amplification primers and buffer by inversion after thawing.

3.3.2 The reaction system was prepared according to table 8 in the 0.2 mL PCR tube with purified ligation product.

TABLE 8

| Component | Volume | |
|---|---|---|
| Purified adapter-ligated product | 20 | μL |
| Library Construction Amplification Primers | 5 | μL |
| (SEQ ID NO: 1-SEQ ID NO: 2) (10 μM) | | |
| Library Construction Amplification | 25 | μL |
| Mixture | | |
| Total Volume | 50 | μL |

3.3.3 The system was mixed by gentle pipetting up and down. Do not mix by vortexing. Centrifuge briefly to ensure the content is collected at the bottom of the tube Amplification was performed according to the procedure shown in table 9.

TABLE 9

| | | |
|---|---|---|
| Temperature | Time | Cycle number |

| | | |
|---|---|---|
| Heated Lid (105° C.) | Turn On | NA |

| | | | | |
|---|---|---|---|---|
| 98° | C. | 3 | min | 1 |
| 98° | C. | 20 | sec | 5-13* |
| 60° | C. | 15 | sec | |
| 72° | C. | 30 | sec | |
| 72° | C. | 5 | min | 1 |

| | | | |
|---|---|---|---|
| 4° | C. | Hold | 1 |

Except the heated lid, the library amplification reaction procedure listed in table 9 can be described specifically as follows:
(1) initial denaturation at 98° C. for 3 minutes;
(2) denaturation at 98° C. for 20 seconds;
(3) annealing at 60° C. for 15 seconds;
(4) extension at 72° C. for 30 seconds;
(5) final extension at 72° C. for 5 minutes;
wherein the cycle number of steps (2)-(4) were 5-13.

In table 9, the cycle number labeled by * was the total cycle number of steps (2)-(4). An appropriate cycle number was recommended according to the input amount of the sample DNA in order to obtain enough library for enrichment. Specific recommendations were as follows:

It is recommended that at least 7 PCR cycles were required for 50 ng input amount of gDNA, and for each doubling of the input, the number of PCR cycles decreases by one. At least 3 PCR cycles were required for 500 ng input amount of gDNA. That is, the total cycle number of steps (2)-(4) are at least 7 when the total input amount of DNA extracted from the sample to be detected is 50 ng, and the cycle number of steps (2)-(4) are at least 3 when the total input of DNA extracted from the sample to be detected is 500 ng.

3.5 Post-Amplification Purification

Agencourt AMPure XP Beads were recommended for purification.

3.5.1 Magnetic beads were equilibrated at room temperature at least 30 minutes and thoroughly homogenized by vortexing, and 50 μL of the magnetic beads was transferred into a new 1.5 mL centrifuge tube.

3.5.2 50 μL of the PCR product was transferred into the 1.5 mL centrifuge tube prepared in step 3.5.1, mixed by vortexing, and incubate at room temperature for 5 min.

3.5.3 Place the 1.5 mL centrifuge tube on the magnetic rack, and incubate until the solution is completely clear, and then remove and discard the supernatant without disturbing magnetic beads.

3.5.4 200 μL of freshly prepared 80% ethanol was added followed by incubation at room temperature for 30 sec, and the supernatant was discarded.

3.5.5 Step 3.5.4 was repeated once.

3.5.6 The 1.5 mL centrifuge tube from step 3.5.5 was centrifuged briefly and placed on the magnetic rack for 1 min, and all the residual solution was discarded, and the lid was opened for drying at room temperature until ethanol was completely evaporated. (Do not over dry the beads, which may result in reduced yield when beads are cracked.).

3.5.7 Resuspend beads with 32 μL of Nuclease-Free Water, mix by vortexing, incubate at room temperature for 2 min, centrifuge briefly and place the tube on the magnetic rack. After the solution was completely clear, 30 μL of supernatant was transferred into a new labeled 1.5 mL centrifuge tube. Note: the gDNA library should be stored at −25° C.-−15° C. if the next step was not proceeded immediately.

3.6 Library Quality Control

The concentration of the gDNA library was quantified using the nucleic acid quantification kit (Qubit™ dsDNA HS Assay Kit) with matched equipment. The total amount of gDNA library should be ≥500 ng, otherwise, the prepared library would be failed to pass the quality control test, and re-construction of library should be required.

3.6.2 The gDNA library was sized using DNA High Sensitivity Reagent Kit with matched equipment. The peak size of gDNA library should be located between 200 and 700 bp and there should be no obvious peaks outside of 200-700 bp, otherwise, the prepared library would be failed to pass the quality control test, and re-construction of library should be required.

step (II), capture library preparation. The gDNA library hybridization capture was done using the probe pool, the captured product was amplified and purified to get captured libraries. The detailed steps are described as follows:

1. Pool and Dry Down Libraries

1.1 Multiple libraries were combined into a pool in a new 1.5 mL centrifuge tube.

It was recommended to mix 4 DNA libraries with similar amplification efficiency (500 ng per library was recommended) into a pool (the total amount of each pool was between 1000 and 2000 ng). It was recommended to mix the DNA positive control and DNA negative control libraries into one pool.

1.2 Prepare blocker mixture according to table 10 in a new 1.5 mL centrifuge tube.

TABLE 10

| Component | Volume |
|---|---|
| Blocker agent 1 | 1 μL |
| Blocker agent 2 | 5 μL |
| Total Volume | 6 μL |

1.3 After well mixing, 6 μL of blocker mixture was added to each pool.

1.4 After thorough mixing by vortex, the liquid was centrifuged briefly to ensure the liquid is collected at the bottom of the tube. Open the lid.

1.5 Place the tube in a vacuum concentrator to dry down the mixture.

2. Library Resuspension, Denaturation, and Hybridization Capture

2.1 The hybridization reaction solution was prepared according to table 11 in a new 1.5 mL centrifuge tube, mixed by vortexing and centrifuged for later use.

TABLE 11

| Component | Volume | |
|---|---|---|
| Hybridization buffer 1 | 8.5 | μL |
| Hybridization buffer 2 | 2.7 | μL |
| Nuclease-Free Water | 3.5 | μL |
| Probe library (0.75 pmol/μL) | 1.3 | μL |
| Total Volume | 17 | μL |

2.2 17 μL of the hybridization reaction solution was added. After thoroughly mixing by vortexing, the solution was centrifugated briefly to ensure the liquid is collected at the bottom of the tube, and then placed at room temperature in the dark for 10 min.

2.3 All 17 μL of liquid was transferred into a new labeled 0.2 mL PCR tube. Then centrifuge briefly to ensure the liquid is collected at the bottom of the tube. Hybridization was performed according to the procedure listed in table 12.

TABLE 12

| Temperature | Time | |
|---|---|---|
| Heated Lid (105° C.) | Turn on | |
| 95° C. | 30 | sec |
| 65° C. | 12~16 | h |

| | |
|---|---|
| 65° C. | Hold |

3. Buffer Preparation & M270 Streptavidin Magnetic Bead Washing

Dynabeads® M270 Streptavidin (M270 streptavidin magnetic beads) from Thermo Fisher Scientific was recommended for capture.

3.1 Turn on the dry bath and the temperature was set at 65° C.

3.2 The M270 streptavidin magnetic beads were equilibrated at room temperature for 30 min.

3.3 The stock solution listed in the table was thawed at room temperature and homogenized by inversion. A working wash buffer was prepared according to table 13.

TABLE 13

| Working wash | Volume of stock | Nuclease-Free | | Total | |
|---|---|---|---|---|---|
| buffer | solution | Water volume | | volume | |
| MagW | Bead wash buffer: 40 μL | 40 | μL | 80 | μL |
| W1-1 | Wash Buffer 1*: 16 μL | 144 | μL | 160 | μL |
| W1-2 | Wash Buffer 1*: 11 μL | 99 | μL | 110 | μL |
| W2 | Wash Buffer 2: 16 μL | 144 | μL | 160 | μL |
| W3 | Wash Buffer 3: 16 μL | 144 | μL | 160 | μL |
| W4 | Wash Buffer 4: 32 μL | 288 | μL | 320 | μL |

If the Wash Buffer 1 is cloudy, heat the bottle in a 65° C. dry bath to allow resuspension.

3.4 W1-2 and W4 were preheated to 65° C. in a 65° C. dry bath for later use.

3.5 The magnetic bead resuspension buffer was prepared according to table 14 in a new 1.5 mL centrifuge tube.

TABLE 14

| Component | Volume | |
|---|---|---|
| Hybridization buffer 1 | 8.5 | μL |
| Hybridization buffer 2 | 2.7 | μL |
| Nuclease-Free Water | 5.8 | μL |
| Total Volume | 17 | μL |

3.6 The M270 streptavidin magnetic beads were homogenized by vortexing for about 1 min. 10 μL beads per pool were added to a new 1.5 mL centrifuge tube.

3.7 20 μL of mixed MagW per pool was added and gently pipette mix10 times. Place the tube on a magnetic rack for 1 min or until the supernatant is completely clear. The supernatant was discarded without disturbing the magnetic beads.

3.8 Step 3.7 was repeated twice.

3.9 17 μL of the magnetic bead resuspension buffer per pool was added to resuspend the magnetic beads. The resuspension solution was centrifuged briefly to ensure the solution is collected at the bottom of the tube, and transferred to a new labeled 0.2 mL PCR tube. The PCR tube was preheated for 2 min on a PCR Thermal Cycler set according to the reaction procedure listed in table 15.

TABLE 15

| Temperature | Time |
|---|---|
| Heated lip (70° C.) | Turn on |
| 65° C. | Hold |

4. Capture and Wash

4.1

4.2 All the preheated 17 μL magnetic beads were transferred into the 0.2 mL PCR tube containing hybridization reaction mixture, and mixed by vortexing, and then placed into the PCR Thermal Cycler which is set according to the reaction procedure in 3.9.

4.3 Incubation was performed for 45 min, with resuspending the magnetic beads every 12 min.

4.4 After the incubation, 100 μL of pre-heated W1-2 was added into the sample tube and mixed by pipetting for 10 times, avoiding formation of air bubbles. The entire reaction were transferred into a new pre-heated 1.5 mL centrifuge tube.

4.5 After mixing by vortexing for 5 sec and centrifuging briefly, place the 1.5 mL centrifuge tube on a magnetic rack until the solution is completely clear, and the supernatant was discarded.

4.6 150 μL of pre-heated W4 was added to the sample tube, followed by vortexing for 5 sec, being careful to not introduce air bubbles. All the mixture was transferred into a new 1.5 mL pre-heated centrifuge tube.

4.7 Incubate in a dry bath at 65° C. for 5 minutes. After mixing by vortexing for 5 sec and centrifuging briefly, place the 1.5 mL centrifuge tube on the magnetic rack until the solution is completely clear, and the supernatant was discarded.

4.8 Steps 4.6 and 4.7 were repeated once.

4.9 150 μL of W1-1 was added to the 1.5 mL centrifuge tube and mixed by vortexing.

4.10 Incubate for 2 min at room temperature while alternating between vortexing for 30 sec and resting for 30 sec.

4.11 Centrifuge briefly, place the 1.5 mL centrifuge tube on the magnetic rack until the solution became completely clear, and the supernatant was discarded.

4.12 150 μL of W2 was added to the 1.5 mL centrifuge tube and mixed by vortexing.

4.13 Incubate for 2 min at room temperature while alternating between vortexing for 30 sec and resting for 30 sec.

4.14 Centrifuge briefly, place the 1.5 mL centrifuge tube on the magnetic rack until the solution became completely clear, and the supernatant was discarded.

4.15 150 μL of W3 was added into the 1.5 mL centrifuge tube followed by vortexing.

4.16 Incubate for 2 min at room temperature while alternating between vortexing for 30 sec and resting for 30 sec.

4.17 Centrifuge briefly, place the 1.5 mL centrifuge tube on the magnetic rack until the solution became completely clear, and the supernatant was discarded.

4.18 After centrifugation, all W3 residue was discarded.

4.19 20 μL of Nuclease-Free Water was added into the sample tube. The magnetic beads were resuspended by pipetting 10 times, and all the resuspended solution was transferred into a new labeled 0.2 mL PCR tube.

So far, the respective capture products were obtained from the gDNA library.

5. Amplification of Capture Product

5.1 A mix for captured library amplification was prepared according to table 16 in a new 1.5 mL centrifuge tube.

TABLE 16

| Component | Volume | |
|---|---|---|
| Captured Library Amplification Mixture | 25 | μL |
| Captured Library Amplification Primers | 2.5 | μL |
| (SEQ ID NO: 3-SEQ ID NO: 4) (10 μM) | | |
| Nuclease-Free Water | 2.5 | μL |
| Total Volume | 30 | μL |

5.2 30 μL of the mix for captured library amplification was transferred into the 0.2 mL PCR tube in 4.19. Mix by vortexing and centrifuge briefly, the 0.2 mL PCR tube was placed into the PCR Thermal Cycler and run the program listed in table 17.

TABLE 17

| Temperature | | Time | | Number of cycles |
|---|---|---|---|---|
| Heated Lid at 105° C. | | On | | NA |
| 98° | C. | 45 | sec | 1 |
| 98° | C. | 15 | sec | Amplification cycle for |
| 60° | C. | 30 | sec | captured libraries is 11~13 * |
| 72° | C. | 30 | sec | |
| 72° | C. | 1 | min | 1 |

| | | | |
|---|---|---|---|
| 4° | C. | Hold | 1 |

*The reaction procedure for captured library amplification listed in table 19; except the step of heated lid; is specifically described as follows:
(1) initial denaturation at 98° C. for 45 seconds;
(2) denaturation at 98° C. for 15 seconds;
(3) annealing at 60° C. for 30 seconds;
(4) extension at 72° C. for 30 seconds;
(5) final extension at 72° C. for 1 minute,
wherein for amplification of captured gDNA libraries, the total cycles of steps (2)-(4) are 11-13.

In table 17, the cycle number indicated by * was the cycle number for repeating steps (2)-(4). An appropriate cycle number was determined according to the input of libraries for capture to obtain enough captured libraries for sequencing.

6 Purification after Amplification

Agencourt AMPure XP Beads were recommended for purification.

6.1 Magnetic beads were equilibrated at room temperature at least 30 minutes and thoroughly homogenized by vortexing, and 75 μL of the magnetic beads was transferred into a new 1.5 mL centrifuge tube.

6.2 All the captured amplification products were transferred into the 1.5 mL centrifuge tube in step 6.1, mixed by vortexing, and incubated at room temperature for 5 min.

6.3 Place the 1.5 mL centrifuge tube on the magnetic rack until the solution become completely clear, and the supernatant was discarded without disturbing the magnetic beads.

6.4 200 μL of freshly prepared 80% ethanol was added followed by incubation at room temperature for 30 sec, and the supernatant was discarded.

6.5 Steps 6.4 were repeated once.

6.6 The 1.5 mL centrifuge tube was centrifuged briefly and placed on the magnetic rack for 1 min, and the residual solution was discarded, and the cap was opened for drying at room temperature until ethanol was completely evaporated. Do not over dry the beads, which may result in reduced yield when beads are cracked

6.7 22 μL of Nuclease-Free Water was added followed by vortexing. Incubate at room temperature for 2 min, centrifuge briefly and place on the magnetic rack. After the solution is completely clarified, 20 μL of supernatant was transferred into a new labeled 1.5 mL centrifuge tube.

7. Capture Library Quality Control

7.1 The concentration of the capture libraries was determined using nucleic acid quantification kit (Qubit™ dsDNA HS Assay Kit) with matched equipment. The total amount of the library should be ≥5 ng, otherwise, the library is unqualified, and a re-hybridization capture would be required. If the total amount of the re-captured library is unqualified, the assay should be terminated.

7.2 The captured gDNA library was size using DNA High Sensitivity Reagent Kit with matched equipment. The main peak size should be located between 200 and 700 bp, and there should be no obvious peaks located outside of 200-700 bp, otherwise, the captured library would be failed to pass the quality control test, and re-hybridization capture would be required. If the re-captured library is unqualified, the assay should be terminated.

So far, the captured gDNA library was obtained.

Step (III), sequencing: the captured library is sequenced by high-throughput sequencing to obtain sequencing data.

Step (IV), Data Analysis

Genomic alterations, including single base substitutions (SNVs), short and long indels, copy number variations (CNVs), and gene rearrangements and fusions, were assessed. Raw reads are aligned with a human genome reference sequence (hg19) using Burrows-Wheeler Aligner, followed by PCR deduplication using Picard's MarkDuplicates algorithm. Read depth is less than 30×, variants of which strand bias was greater than 10% or VAF was less than 0.5% were removed. Common single nucleotide polymorphisms (SNPs) defined as either from the dbSNP database (version 147) or with a frequency exceeding 1.5% of the Exome Sequencing Project 6500 (ESP6500) or more than 1.5% of the 1000 Genomes Project were also excluded.

Whether the identified mutation is true is judged by the following criteria:
(1) for point mutation:

The sequencing coverage depth at the location of the point mutation is greater than 500 times; the quality score of each read containing the point mutation is greater than 40, and the quality score of the base corresponding to the point mutation on each read containing the point mutation is greater than 21; the number of reads containing the point mutation is greater than or equal to 5; the ratio of forwarding read length to reverse read length of all reads containing the point mutation is less than ⅙; the variant allele frequency of tumor tissue/the variant allele frequency of control tissue is greater than or equal to 20;
(2) for indel:
if there are less than 5 consecutive identical bases in the indel, the sequencing coverage depth at the location of the indel is greater than 600 times; the quality score of each read containing the indel is greater than 40, and the quality score of the base corresponding to the indel mutation on each read containing the indel is greater than 21; the number of reads containing the indel is greater than or equal to 5; the ratio of forward read length to reverse read length of all reads containing the indel is less than ⅙; the variant allele frequency of tumor tissue/the variant allele frequency of control tissue is greater than or equal to 20;
if there are greater than and equal to 5 and less than 7 consecutive identical bases in the indel, the sequencing coverage depth at the location of the indel is greater than 60 times; the quality score of each read containing the indel is greater than 40, and the quality score of the base corresponding to the indel mutation on each read containing the indel is greater than 21; the number of reads containing the indel is greater than or equal to 5; the ratio of forward read length to reverse read length of all reads containing the indel is less than ⅙; the variant allele frequency of tumor tissue/the variant allele frequency of control tissue is greater than or equal to 20; the variant allele frequency of the tumor tissue is greater than or equal to 10%;
if there are greater than 7 consecutive identical bases in the indel, the sequencing coverage depth at the location of the indel is greater than 60 times; the quality score of each read containing the indel is greater than 40, and the quality score of the base corresponding to the indel mutation on each read containing the indel is greater than 21; the number of reads containing the indel is greater than or equal to 5; the ratio of forward read length to reverse read length of all reads containing the indel is less than ⅙; the variant allele frequency of tumor tissue/the variant allele frequency of control tissue is greater than or equal to 20; the variant allele frequency of the tumor tissue is greater than or equal to 20%.
(3) Amplification mutation

Amplification mutation refers to a type of variation varied in gene copy number. Amplifications are CNVs with increased copy number. CNV, namely, copy number variation, generally refers to duplication and deletion in the copy number of large genomic fragments ranging from 1 kb to several Mb in length.

TMB Calculation

In addition to routine detection of genomic alterations, TMB was also determined by NGS-based algorithms. TMB was estimated by counting somatic mutations including SNVs and indels per megabase of the coding region sequence examined. Driver gene mutations and known germline alterations in dbSNPs were excluded.

Immunohistochemistry

Immunohistochemical (IHC) staining process was performed as previously described. Briefly, samples were deparaffinized, rehydrated, and target-recovered, and then incubated with monoclonal antibodies against PD-L1 (DAKO, clones 22C3 and 28-8). The slides were incubated with ready-to-use chromogenic reagents consisting of secondary antibody molecules and horseradish peroxidase (HRP) molecules coupled to the dextran polymer backbone. Subsequent enzymatic conversion upon addition of a chromophore and enhancer results in visible reaction products precipitated at the antigenic site. The samples were then counterstained with hematoxylin.

A cohort of data acquired from public database In order to further verify the clinical predictive effect of variations in four members of the NOTCH family (NOTCH1, NOTCH 2, NOTCH 3, and NOTCH 4) on immune checkpoint inhibitor therapy, data, including patient clinical baseline data, data for evaluating the efficacy of immune checkpoint inhibitor treatment, and patient genomic data, from a cohort of 1,661 cases with solid tumors were downloaded from cBioPortal, a database for the tumor genomics, on the website http://www.cbioportal.org.

In the above detection process,

(1) When the library is constructed, end repairing and addition of poly-A tail are followed by the ligation with a adaptor and end repairing and addition of poly-A tail again, and there is only one purification process. This reduces time and steps for manual operation as well as the overall process time. Moreover, the loss of template diversity which will result from the increase of purification times will be reduced in one purification process;

(2) In the whole process, the input amount (the sample load) can affect the success rate of the detection. The standard of sample load was set from 50 ng to 1000 ng by the laboratory, to make ensure that the acquired library can be present at an appropriate amount. The inventor recommends that the recommended amount of each library is 500 ng to ensure a certain success rate of the detection and detection consistency. Therefore, it is necessary to determine the appropriate sample load; thus, for the DNA extracted from the sample to be tested, the total amount of nucleic acid extracted is recommended to be at least 50 ng and at most 500 ng. In the case of a given sample load, the number of PCR amplification cycles for the above (2)-(4) in the library amplification process most directly affects the yield of the library. Too many PCR cycles will lead to increased bias in the library amplification process, while too few PCR cycles will result in insufficient library yield, which cannot meet the requirement for subsequent quality control and on-board sequencing. However, in this example, the number of cycles (of steps (2)-(4)) of the library amplification process is relatively small (the above number of cycles for 50 ng DNA can be as few as 7 times, and the number of cycles for 500 ng DNA can be as few as 3 times). This can avoid the generation of duplicate (Duplicate reads will be deduplicated during analysis with only unique reads retained) at the raw signal end due to the large number of cycles, so as to avoid a small amount of data distributed in the target area at the same amount of sequencing data, thereby improving the accuracy of the detection results. Similarly, the cycle numbers of (2)-(4) in the capture amplification in this example also ensure a small number.

### Example 1 Characteristics of Patients

A total of 4,596 Chinese patients with solid tumors participated in this study. Among the 4,596 patients, various types of tumors in the patients were as follows: 2,859 cases of lung adenocarcinoma (62.1%), 406 cases of lung squamous cell carcinoma (8.8%), 141 cases of small cell lung cancer (3.1%), 639 cases of hepatocellular carcinoma (14%), 254 cases of esophageal tumor (5.5%), 174 cases of breast cancer (3.8%), 74 cases of head and neck cancer (1.7%), 44 cases of endometrial cancer (1%), and 5 cases of squamous cell carcinomas (0.1%).

The characteristics of the patients are shown in table 18. Most patients with a NOTCH family gene mutant were male (73.2% vs 57.7%, p<0.001), and the median age at diagnosis for patients with a NOTCH family gene mutant was 61 years. TMB in 4,596 patients were tested. The median TMB in the overall population was 5.4 muts/Mb.

TABLE 18
Characterization of Patients

| | Overall | | NOTCH1/2/3/4-WT | | NOTCH1/2/3/4-MUT | | |
|---|---|---|---|---|---|---|---|
| | (n = 4596) | | (n = 4152) | | (n = 444) | | p value |
| Gender | | | | | | | <0.001 |
| Male | 2723 | (59.2%) | 2398 | (57.7%) | 325 | (73.2%) | |
| Female | 1873 | (40.8%) | 1754 | (42.2%) | 119 | (26.8%) | |
| Age | | | | | | | 0.001 |
| Mean (SD) | 56.7 | (16.0) | 56.5 | (16.1) | 58.4 | (14.6) | |
| Median (Min, Max) | 59.0 | [0.00, 200] | 59.0 | [0.00, 200] | 61.0 | [0.00, 84.0] | |
| TMB | | | | | | | <0.001 |
| Mean (SD) | 7.57 | (9.72) | 6.78 | (7.98) | 15.0 | (18.0) | |
| Median [Min, Max) | 5.40 | [0.00, 215] | 4.60 | [0.00, 122] | 10.0 | [0.00, 215] | |
| Type of tumors | | | | | | | <0.001 |
| Lung squamous cell carcinoma | 406 | (8.8%) | 334 | (8.0%) | 72 | (16.2%) | |
| Lung adenocarcinoma | 2859 | (62.2%) | 2673 | (64.4%) | 186 | (41.9%) | |
| Hepatocellular carcinoma | 639 | (13.9%) | 594 | (14.3%) | 45 | (10.1%) | |
| Invasive breast cancer | 174 | (3.8%) | 159 | (3.8%) | 15 | (3.4%) | |
| Skin tumor | 5 | (0.1%) | 3 | (0.1%) | 2 | (0.5%) | |
| Esophageal tumor | 254 | (5.5%) | 176 | (4.2%) | 78 | (17.6%) | |
| Head and neck tumor | 74 | (1.6%) | 65 | (1.6%) | 9 | (2.0%) | |
| Small cell lung cancer | 141 | (3.1%) | 113 | (2.7%) | 28 | (6.3%) | |
| Endometrial cancer | 44 | (1.0%) | 35 | (0.8%) | 9 | (2.0%) | |

Example 2 Frequency of pathogenic mutations in four NOTCH family genes (NOTCH1-4) in Chinese population with solid tumors and their correlation with immunotherapy biomarker TMB

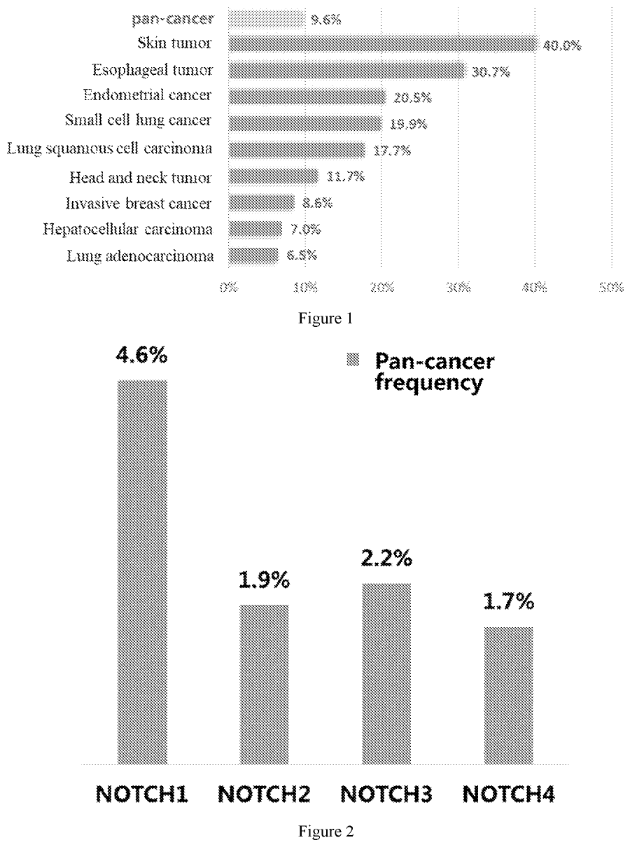

An overall mutation rate of NOTCH family genes (NOTCH1/2/3/4) in the Chinese population was 9.6% (FIG. 1). Among them, esophageal tumors (30.7%), endometrial cancer (20.5%), small cell lung cancer (19.9%), lung squamous cell carcinoma (17.7%), and head and neck tumors (11.7%) had higher mutation rates.

The mutation rates of NOTCH1, NOTCH2, NOTCH3, and NOTCH4 in Chinese patients with solid tumors were 4.6%, 1.9%, 2.2%, and 1.7%, respectively (FIG. 2).

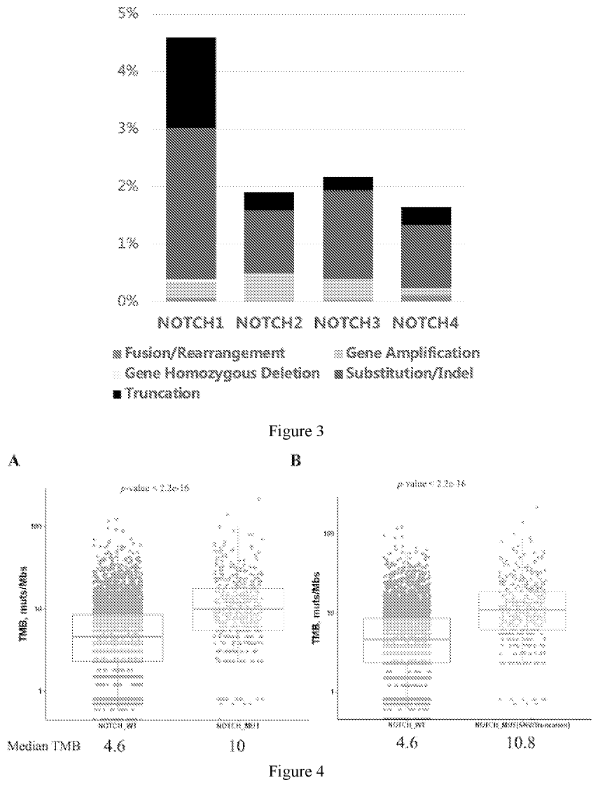

The mutation frequencies of the main variant forms of the four NOTCH family genes (NOTCH1-4) are shown in FIG. 3: for NOTCH1, the mutation frequency was 2.6% for point mutations, 1.6% for truncation variations, 0.3% for amplification, and 0.3% for fusions/rearrangements; for NOTCH2, the mutation frequency was 1.1% for point mutations, 0.3% for truncation variations, and 0.5% for amplification; for NOTCH3, the mutation frequency was 1.5% for point mutations, 0.2% for truncation variations, and 0.4% for amplification; for NOTCH4, the mutation frequency was 1.1% for point mutations, 0.3% for truncation variations, 0.1% for amplification, and 0.1% for fusions/rearrangements.

For all types of tumors, patients with variations in four NOTCH family genes (NOTCH1-4) had significantly higher TMB than wild-type (median TMB: 10 vs. 4.6, p<0.001) (FIG. 4A). Patients with point mutations or truncation mutations in four NOTCH family genes (NOTCH1-4) had significantly higher TMB than wild-type (median TMB: 10.8 vs. 4.6, p<0.001) (FIG. 4B).

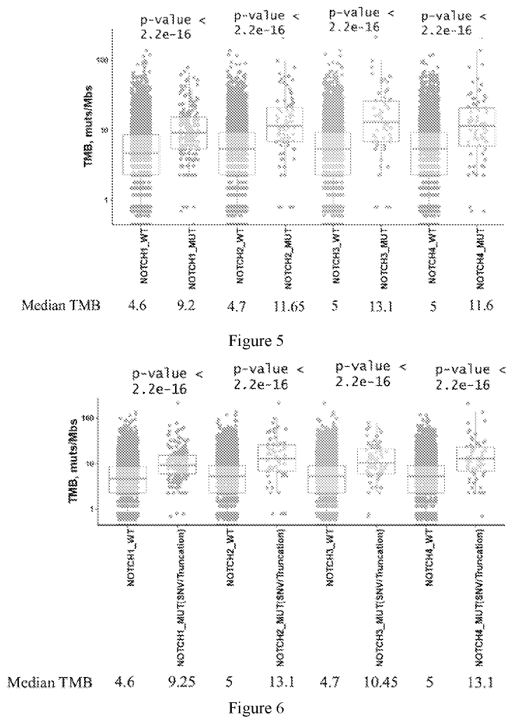

For all types of tumors, patients with a variation in NOTCH1 gene had significantly higher TMB than wild-type (median TMB: 9.2 vs. 4.6, p<0.001); patients with a variation in NOTCH2 gene had significantly higher TMB than wild-type (median TMB: 11.65 vs. 4.7, p<0.001); patients with a variation in NOTCH3 gene had significantly higher TMB than wild-type (median TMB: 13.1 vs. 5, p<0.001); patients with a variation in NOTCH4 gene had significantly higher TMB than wild-type (median TMB: 11.6 vs. 5, p<0.001) (FIG. 5).

Patients with point mutations or truncation mutations in NOTCH1 gene had significantly higher TMB than wild-type (median TMB: 9.25 vs. 4.6, p<0.001); patients with point mutations or truncation mutations in NOTCH2 gene had significantly higher TMB than wild-type (median TMB: 13.1 vs. 5, p<0.001); patients with point mutations or truncation mutations in NOTCH3 gene had significantly higher TMB than wild-type (median TMB: 10.45 vs. 4.7, p<0.001); patients with point mutations or truncation mutations in NOTCH4 gene had significantly higher TMB than wild-type (median TMB: 13.1 vs. 5, p<0.001) (FIG. 6).

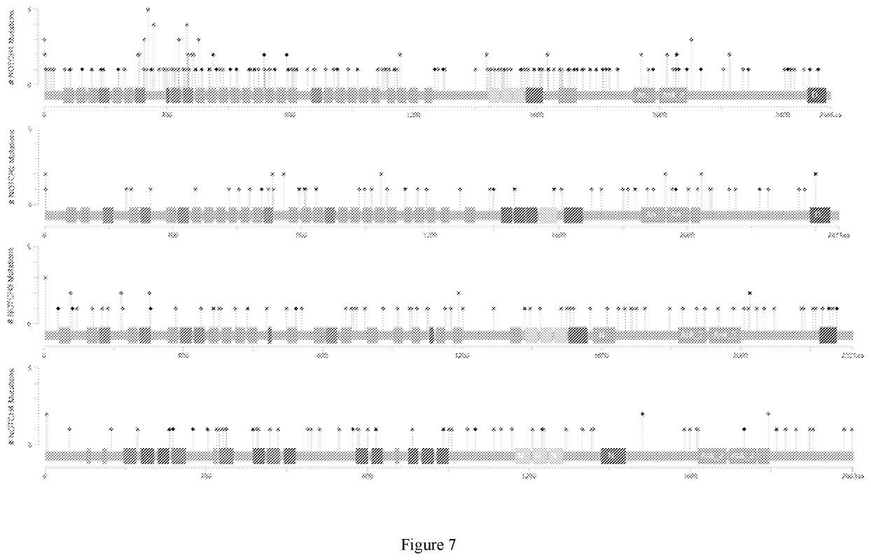

The sites of the mutation in four NOTCH family genes (NOTCH1-4) were scattered, and there was no obvious hot-spot mutation region (FIG. 7).

Example 3 Clinical data validation of variations in four NOTCH family genes (NOTCH1-4) as immunotherapy biomarkers

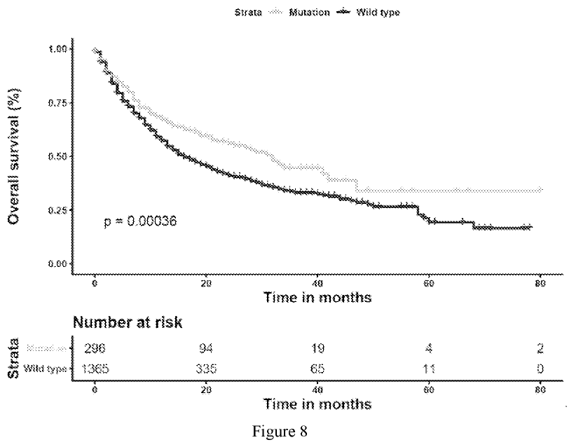

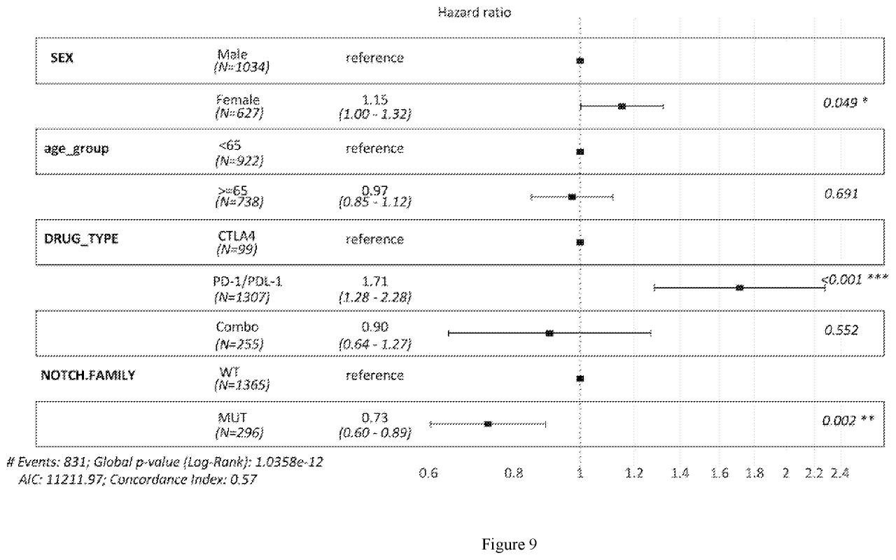

To further validate the value of mutations in four NOTCH family genes (NOTCH1-4) for predicting treatment with immune checkpoint inhibitors (ICIs), external validation was performed by downloading cohort information from public databases. The cohort data uploaded by Robert M. Samstein et al. were downloaded from the cBioPortal website (http://www.cbioportal.org/). The Robert M. Samstein cohort included 1,661 patients with solid tumors who received therapy by using anti-PD-(L)1 alone or in combination with anti-CTLA-4. The specific data about patient baseline can be found in the literature (see Samstein R M, Lee C-H, Shoushtari A N et al. Tumor mutational load predicts survival after immunotherapy across multiple cancer types. Nature genetics 2019). Among the 1,661 patients, 296 patients (17.8%) had mutations in four NOTCH family genes (NOTCH1-4). The median overall survival time (median OS) of patients in NOTCH family four-gene (NOTCH1-4) variant group after receiving immunotherapy was longer than that of NOTCH family (NOTCH1-4) four-gene wild-type patients (median OS: 32 vs. 16 months, p<0.001) (FIG. 8). The results of Cox multivariate analysis further indicated that variations in the four NOTCH family-genes (NOTCH1-4) was an independent predictive risk factor for the prognosis after immunotherapy (HR: 0.73, 95% CI: 0.6-0.89, p=0.002) (FIG. 9).

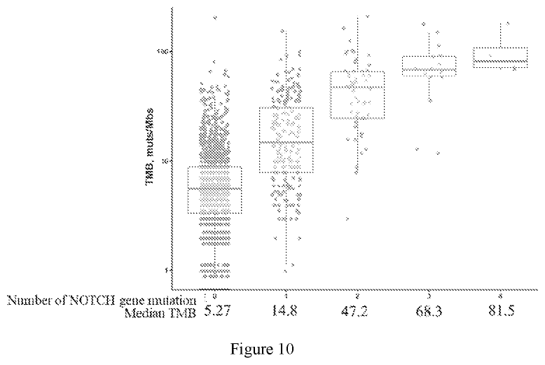

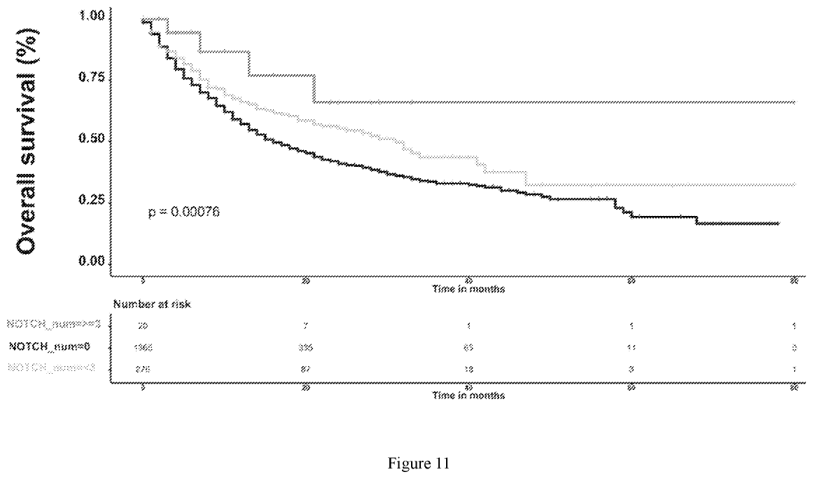

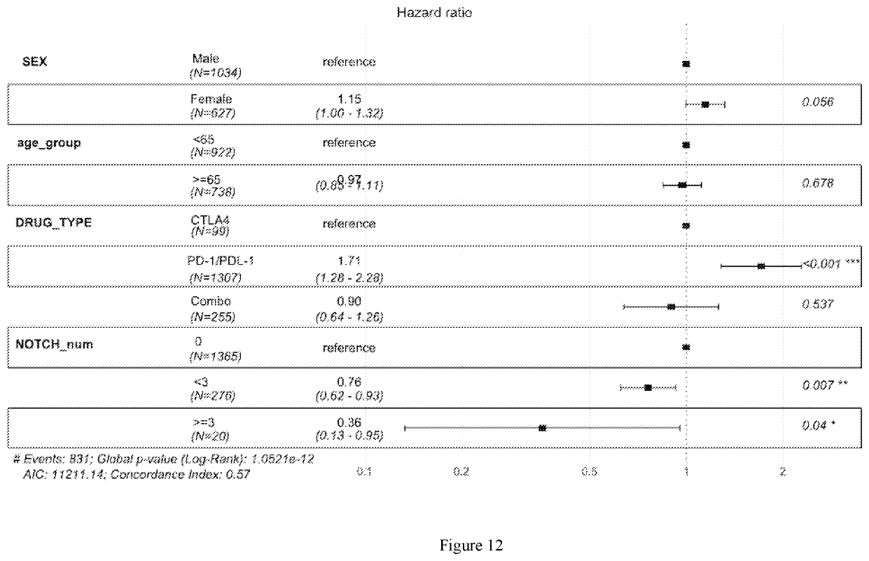

The correlation between the number of the mutant NOTCH family genes and the efficacy of immunotherapy was further analyzed. In the Robert M. Samstein cohort, 1,365 cases were NOTCH family protein wild type (the number of mutants was 0), 217 cases had one mutant NOTCH family gene (the number of mutants was 1), 59 cases had two mutant NOTCH family genes (the number of mutants was 2), 16 cases had three mutant NOTCH family genes (the number of mutants was 3), 4 cases had four mutant NOTCH family genes (the number of mutants was 4). As the number of mutant NOTCH genes increased, TMB gradually increased, and the TMBs in groups having 3 or 4 mutants were significantly higher than that of the other three groups (p<0.05) (FIG. 10). Similarly, the results of survival analysis also showed that the median OS of the groups with more than or equal to three mutant NOTCH genes was significantly longer than that of the groups with less than 3 mutations and without mutant NOTCH genes (p<0.001) (FIG. 11). The median OS of the groups with three or more mutant NOTCH genes was NA (95% CI: 21-NA). The median OS of the group with less than three mutant NOTCH genes was 31 (95% CI: 22-47). The median OS of the group without mutant NOTCH genes (wild type) was 16 (95% CI: 15-19). It was further confirmed by COX multivariate regression analysis that the number of the mutant NOTCH family genes was an independent predictor of immunotherapy. The groups with greater than or equal to three mutant NOTCH genes vs the group without mutant NOTCH genes (wild type): HR: 0.36, 95% CI: 0.13-0.95, p=0.04; The groups with less than three mutant NOTCH genes vs the group without mutant NOTCH genes (wild type): HR: 0.76, 95% CI: 0.62-0.93, p=0.007 (FIG. 12).

In addition, the present disclosure provides the following embodiments:

Embodiment 1: Use of a detection agent for detecting NOTCH family gene variations in preparing a kit for predicting sensitivity to an immune checkpoint inhibitor therapy in a patient with a solid tumor, wherein the presence of the NOTCH family gene variations is an indication that the patient with a solid tumor is sensitive to immune checkpoint inhibitor therapy;

the NOTCH family gene is at least one selected from the group consisting of NOTCH1, NOTCH2, NOTCH3, and NOTCH4.

Embodiment 2: Use of a detection agent for detecting NOTCH family gene variations in preparing a kit for predicting a degree of tumor mutation burden in a subject with a solid tumor, wherein the presence of the NOTCH family gene variations is an indication of high tumor mutation burden.

The NOTCH family gene is at least one selected from the group consisting of NOTCH1, NOTCH2, NOTCH3, and NOTCH4.

Embodiment 3: The use according to claim 1 or 2, wherein the immune checkpoint inhibitor is a PD-1 inhibitor and/or a PD-L1 inhibitor.

Embodiment 4: The use according to claim 1 or 2, wherein the gene variations includes one or more of point mutations, truncation mutations, amplification variations, and fusion/rearrangement.

Embodiment 5: The use according to claim 4, wherein the gene variations includes point mutations and truncation mutations.

Embodiment 6: The use according to claim 1 or 2, wherein the detection agent is for detecting at the nucleic acid level.

Embodiment 7: The use according to claim 6, wherein the detection agent is used to perform any of the following methods:
restriction fragment length polymorphism method, single-strand conformation polymorphism method, polymerase chain reaction, competitive allele-specific PCR, denaturing gradient gel electrophoresis, allele-specific PCR, nucleic acid sequencing, nucleic acid typing on-chip assay, flight mass spectrometry analysis, denaturing high performance liquid chromatography, Snapshot method, Taqman probe method, in situ hybridization, biological mass spectrometry and HRM method.

Embodiment 8: The use according to claim 1 or 2, wherein the detection agent is for detecting at the protein level.

Embodiment 9: The use according to claim 8, wherein the detection agent is used to perform any of the following methods:
biological mass spectrometry, amino acid sequencing, electrophoresis, and detection with specific antibodies designed for mutation sites.

Embodiment 10: The use according to claim 1 or 2, wherein the kit further includes a sample processing reagent including at least one of a sample lysis reagent, a sample purification reagent, and a sample nucleic acid extraction reagent.

Embodiment 11: The use according to claim 10, wherein the sample is at least one selected from blood, serum, plasma, cerebrospinal fluid, tissue or tissue lysate, cell culture supernatant, semen, and saliva samples from the patient with the solid tumor.

The technical features of the embodiments described above may be arbitrarily combined. For the sake of brevity of description, not all possible combinations of the technical features in the aforementioned embodiments are described. However, as long as there is no contradiction between the combinations of these technical features, all should be considered as the scope of this specification.

The above embodiments only represent several examples of the present disclosure, and the description thereof is more specific and detailed, but it should not be construed as restricting the scope of the present disclosure. It should be understood that, the applications of the present disclosure are not limited to the above-described examples, and those skilled in the art can make modifications and changes in accordance with the above description, all of which are within the protection scope of the present disclosure. Therefore, the protection scope of the present disclosure shall be defined by the appended claims.

1. A detection agent for predicting sensitivity to an immune checkpoint inhibitor therapy in a subject with a solid tumor, wherein the detection agent is a detection agent for detecting NOTCH gene variations;
the NOTCH gene is at least one selected from the group consisting of NOTCH1, NOTCH2, NOTCH3, and NOTCH4.

2. The detection agent according to claim 1, wherein the detection agent is a detection agent for detecting the NOTCH gene variations at the nucleic acid level.

3. The detection agent according to claim 1, wherein the detection agent is a detection agent for detecting the NOTCH gene variations at the protein level.

4. The detection agent according to claim 1, wherein the detection agent is a group of DNA probes whose sequence is complementary to the sequence of at least one gene of NOTCH1, NOTCH2, NOTCH3, and NOTCH4.

5. The detection agent according to claim 4, wherein the detection agent is a group of DNA probes whose sequence is complementary to the selected gene sequence from a gene sequence group consisting of Gene ID: 4851, Gene ID: 4853, Gene ID: 4854, and Gene ID: 4855.

6. The detection agent according to claim 4, wherein the immune checkpoint inhibitor is a PD-1 inhibitor and/or a PD-L1 inhibitor.

7. The detection agent according to claim 4, wherein the PD-1 inhibitor is one or more selected from the group consisting of Nivolumab, Pembrolizumab, Jembrolizumab, lambrolizumab, Pidilizumab, Tereprizumab (JS001) and Ipilimumab.

8. The detection agent according to claim 6, wherein the PD-L1 inhibitor is one or more selected from the group consisting of Atezolizumab, JS003, Durvalumab, Avelumab, BMS-936559, MEDI4736, and MSB0010718C.

9. A kit for predicting sensitivity to an immune checkpoint inhibitor therapy in a subject with a solid tumor, comprising the detection agent according to claim 1.

10. The kit according to claim 9, further comprising a DNA negative quality control and a DNA positive quality control, wherein the DNA negative quality control is selected from the group consisting of wild-type NOTCH1, NOTCH2, NOTCH3, NOTCH4 genes, and combinations thereof, and the DNA positive quality control is selected from the group consisting of known NOTCH1, NOTCH2, NOTCH3, NOTCH4 gene variants and combinations thereof.

11. The kit according to claim 10, further comprising a sample processing reagent including at least one of a sample lysis reagent, a sample purification reagent, and a sample nucleic acid extraction reagent.

12. The kit according to claim 11, wherein the sample is at least one selected from blood, serum, plasma, cerebrospinal fluid, tissue or tissue lysate, cell culture supernatant, semen, and saliva samples from the subject with the solid tumor.

13. A method for predicting sensitivity to an immune checkpoint inhibitor therapy in a subject with a solid tumor, comprising: a) using the DNA extracted from a sample to be detected for library preparation to obtain a gDNA library; b) hybridizing the gDNA library with a group of DNA probe for detecting NOTCH gene variations to obtain a hybridization product, wherein the NOTCH gene is at least one selected from the group consisting of NOTCH1, NOTCH2, NOTCH3, and NOTCH4; c) performing amplification and purification after the hybridization capture to obtain a captured gDNA library; d) sequencing the captured gDNA library to obtain targeted gDNA sequencing data; and e) performing bioinformatic analysis of the gDNA sequencing data to obtain information about the NOTCH gene variation.

14. The method according to claim 13, wherein in a), the total amount of the DNA extracted from the sample to be detected is between 50 ng and 500 ng.

15. The method according to claim 13, wherein in a), the extracted DNA is subjected to nucleic acid fragmentation to obtain nucleic acid fragments for constructing the gDNA library.

16. The method according to claim 13, wherein the gDNA library is constructed by following steps: end repair and A-tailing, adapter ligation and post-ligation cleanup, so as to obtain a purified adaptor-ligated product.

17. The method according to claim 13, wherein the detection agent is a group of DNA probes for at least one sequence selected from the following gene sequences: Gene ID: 4851, Gene ID: 4853, Gene ID: 4854, and Gene ID: 4855.

18. The method according to claim 13, wherein the immune checkpoint inhibitor is a PD-1 inhibitor, a PD-L1 inhibitor or a combination thereof.

19. The method according to claim 18, wherein the PD-1 inhibitor is one or more selected from the group consisting of Nivolumab, Pembrolizumab, Jembrolizumab, lambrolizumab, Pidilizumab, Tereprizumab (JS001) and Ipilimumab.

20. The method according to claim 6, wherein the PD-L1 inhibitor is one or more selected from the group consisting of Atezolizumab, JS003, Durvalumab, Avelumab, BMS-936559, MEDI4736, and MSB0010718C.
